(19) United States
(12) Patent Application Publication
(10) Pub. No.: US 2019/0254614 A1
(43) Pub. Date: Aug. 22, 2019

(54) MODULAR CONSOLE ASSEMBLY AND METHODS FOR USE WITH MEDICAL DEVICES

(71) Applicant: BlackHagen-Design, Inc., Dunedin, FL (US)

(72) Inventors: Philip J. D'Almada Remedios, Palm Harbor, FL (US); Bartosz Korec, Palm Harbor, FL (US)

(21) Appl. No.: 16/263,905

(22) Filed: Jan. 31, 2019

Related U.S. Application Data
(60) Provisional application No. 62/632,179, filed on Feb. 19, 2018.

(51) Int. Cl. A61B 5/00

(52) U.S. Cl. CPC A61B 5/7475; A61B 2017/00973; A61B 5/7445

(57) ABSTRACT

A modular medical console system or assembly adaptable for different applications and functions. The assembly includes a console and/or a pedestal stand. The console includes a housing with a pivoting connection for a displace device and a plurality of modular components (e.g., I/O panels, peristaltic pump with patient-circuit cartridge, etc.) for use in any needed medical application. The pedestal stand includes a vertical column with front legs/wheels, rear legs/wheels, and at least one handle disposed substantially around the column. The structural configures of the legs/wheels and handle(s) permit multiple pedestal stands to be nested together.

### CROSS-REFERENCE TO RELATED APPLICATIONS

This nonprovisional application claims priority to U.S. Provisional Patent Application No. 62/632,179, entitled “Modular Console Assembly for Medical Devices,” filed Feb. 19, 2018 by the same inventors, the entirety of each is incorporated herein by this reference.

### TECHNICAL FIELD

The claimed subject matter relates, generally, to medical devices. More specifically, it relates to multi-functional medical consoles. More specifically, to modular multi-functional medical consoles and methods of their use.

### BACKGROUND

Medical consoles are well-known in the art but typically are developed in small volumes during their life cycle. Thus, non-recurring engineering costs associated with these devices (e.g., materials, tooling, design, etc.) are limited due to needs determined by returns on investment.

Accordingly, what is needed is a modular console system or assembly that can be adapted for individual medical applications. However, in view of the art considered as a whole at the time the present invention was made, it was not obvious to those of ordinary skill in the field of this invention how the shortcomings of the prior art could be overcome.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF DESCRIPTION OF THE DRAWINGS

For a fuller understanding of the invention, reference should be made to the following detailed description, taken in connection with the accompanying drawings, in which:

FIG. 1A is a front perspective view of a medical console, according to an embodiment of the current invention, in a collapsed position.

FIG. 1B is alternative perspective view of a medical console in a collapsed position.

FIG. 1C is a perspective view of a medical console in an expanded position.

FIG. 1D is a close-up view of a modular connector piece on a medical console.

FIG. 1E is a rear perspective view of a medical console in an expanded position.

FIG. 1F is an alternative perspective view of a medical console in an expanded position.

FIG. 2A is a side cross-sectional view of a medical console.

FIG. 2B is an exploded view of a medical console.

FIG. 2C is an exploded view of a medical console from an alternative perspective.

FIG. 3A depicts a medical console with small digital device and status indicator, as may be used in a tabletop or rack-mount setting, for example.

FIG. 3B depicts a medical console with a medium-sized digital device.

FIG. 3C depicts a medical console with a large-sized digital device.

FIG. 4A depicts a medical console with a peristaltic pump module with patient-circuit cartridge.

FIG. 4B is a close-up view of the peristaltic pump module.

FIG. 5A is a perspective view of a medical console coupled to a pedestal stand, according to an embodiment of the current invention.

FIG. 5B is an alternative perspective view of a medical console coupled to a pedestal stand.

FIG. 5C is an elevated front view of a pedestal stand.

FIG. 5D is a close-up view of a medical console coupled to a pedestal stand.

FIG. 5E is a close-up view of a medical console disconnected from a pedestal stand.

FIG. 6A is a perspective view of two pedestal stands nested together.

FIG. 6B is a side view of two pedestal stands nested together.

FIG. 7 depicts a medical console in different configurations, including coupled to a pedestal stand, positioned on a tabletop, rack-mount and coupled to an IV pole.

### SUMMARY

This summary is provided to introduce a selection of concepts in a simplified form. These concepts are described in further detail in the detailed description of example embodiments of the disclosure below. This summary is not intended to identify key features or essential features of the claimed subject matter, nor is it intended to be used to limit the scope of the claimed subject matter.

Embodiments disclosed herein include structures of a modular medical console, as well as related devices. For example, in accordance with one embodiment, a modular medical console, comprising: a housing having a front end, a rear end, a left side, a right side, a top side, and a bottom side; a display device connector module that is hingedly connected to the front end of the housing, wherein, a display device is connected to the display device connector module and when the display device has a substantially horizontal position it abuts or is otherwise parallel to the top side of the housing, the display device connected to the display device connector module has an angled position relative to the top side of the housing, such that the angle between the display device and the top side of the housing is more than 0° and less than 360°; and a plurality of modular components removably coupled to the housing.

In additional embodiments, a modular medical console, is portable, is coupled to a pedestal.

In additional embodiments, a modular medical console, wherein the housing is coupled to the pedestal with a toolless connection system.

In additional embodiments, a modular medical console, wherein the pedestal further comprises handles coupled to an upright, wherein the upright is coupled to pedestal base which itself is operably coupled to a set of wheels.

In additional embodiments, a modular medical console, wherein the housing further comprises one or two removable modular adjustable arms disposed on either the left or right side of the housing.

In additional embodiments, a modular medical console, wherein one of the side modules incorporates a peristaltic pump, the peristaltic pump includes a rotor, rollers, and a patient-circuit cartridge that is removably disposed in overlying relation to the rotor and rollers.

In additional embodiments, a modular medical console, wherein the housing further comprises a port.

In additional embodiments, a modular medical console, wherein the port is an input/output panel disposed on the front and/or rear side of the housing.

In additional embodiments, a modular medical console, wherein at least one of the removable modular adjustable arms is a hook able to hold a handpiece, medication bag, or other patient tube-sets.

In additional embodiments, a modular medical console, wherein at least one of the removable modular adjustable arms is capable of holding a first medical tool.

In additional embodiments, a modular medical console, wherein the first medical tool is a diagnostic medical device, a therapeutic medical device, or any other device used for patient care.

In additional embodiments, a modular medical console, further comprising vents on the left, right, or both sides of the housing.

In additional embodiments, a modular medical console, further comprising a generic connector mounting panel coupled to the bottom side of the housing to mount the housing on the generic connector mounting panel.

In additional embodiments, a modular medical console, wherein the generic connector mounting panel includes a top mounting panel, bottom mounting panel, wherein the top mounting panel and bottom mounting panel couple together with a rear mounting panel and connectors.

In additional embodiments, a modular medical console, wherein the housing comprises a second port.

In additional embodiments, a modular medical console, wherein the pedestal comprises a thin, vertical upright column having a front side, a rear side, a left side, a right side, a top end, and a bottom end; wherein the bottom end is coupled to a base. In some embodiments the upright column has a battery pack or other components within it to aid use of the modular medical console.

In additional embodiments, a modular medical console, wherein the base includes a pair of front legs and wheels extending from the vertical column toward the front of the pedestal stand; a pair of rear legs and wheels extending from the vertical column toward the rear of the pedestal stand, wherein the pair of front wheels extend further laterally than the pair of rear wheels, such that a greater distance is present between the pair of front wheels than between the pair of rear wheels, wherein the pair of front legs have a greater height or curvature than the pair of rear legs, wherein the pair of rear legs and wheels is configured to slide under or nest with a second pair of front legs and wheels of a second pedestal stand, whereby the second pair of front legs and wheels of the second pedestal stand is structured similarly to the pair of front legs and wheels of the pedestal stand. In some embodiments the reverse is conceived, wherein the pair of rear wheels extend further laterally than the pair of front wheels, such that a greater distance is present between the pair of rear wheels than between the pair of front wheels, wherein the pair of rear legs have a greater height or curvature than the pair of front legs, wherein the pair of front legs and wheels is configured to slide under or next with a second pair of rear legs and wheels of a second pedestal, whereby the second pair of rear legs and wheels of the second pedestal stand is structured similarly to the pair of rear legs and wheels of the pedestal stand.

In additional embodiments, a modular medical console, wherein the pedestal further comprises a proximal handle coupled to the vertical column and positioned to the front of the vertical column, wherein the proximal handle has a first height off the ground on which the pedestal stand is disposed, a distal handle coupled to the vertical column and positioned to the rear of the vertical column, wherein the distal handle has a second height off the ground on which the pedestal stand is disposed, wherein the second height is greater than the first height, wherein the proximal handle and the distal handle are formed of multiple handles or are formed of a single handle disposed substantially around the vertical column.

In further embodiments, a method of using a modular medical console.

### DETAILED DESCRIPTION

In the following detailed description, reference is made to the accompanying drawings, which form a part thereof, and within which are shown by way of illustration specific embodiments by which the invention may be practiced. It is to be understood that other embodiments may be used, and structural changes may be made without departing from the scope of the present application. These embodiments are described in sufficient detail to enable those of ordinary skill in the art to practice the present disclosure, and it is to be understood that other embodiments may be used, and that structural, logical, and electrical changes may be made within the scope of the disclosure.

From the following descriptions, it should be understood that components of the embodiments as generally described and illustrated in the figures herein could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The following description provides specific details, such as material types, compositions, material thicknesses, and processing conditions in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional techniques employed in the industry. Only those process acts and structures necessary to understand the embodiments of the disclosure are described in detail below. A person of ordinary skill in the art will understand that some process components are inherently disclosed herein and that adding various conventional process components and acts would be in accord with the disclosure. In this description, specific implementations are shown and described only as examples and should not be construed as the only way to implement the present disclosure unless specified otherwise herein.

Illustrations presented herein are not meant to be actual views of any particular material, component, or system, but are merely idealized representations that are employed to describe embodiments of the disclosure. Referring in general to the following description and accompanying drawings, various embodiments of the present disclosure are illustrated to show its structure and method of operation. Common elements of the illustrated embodiments may be designated with similar reference numerals. It should be understood that the figures presented are not meant to be illustrative of actual views of any particular portion of the actual structure or method but are merely idealized representations employed to more clearly and fully depict the present invention defined by the claims below.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

Any headings used herein should not be considered to limit the scope of embodiments of the invention as defined by the claims below and their legal equivalents. Concepts described in any specific heading are generally applicable in other sections throughout the entire specification.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

As used herein, “about” means approximately or nearly and in the context of a numerical value or range set forth means ±15% of the numerical. In an embodiment, the term “about” can include traditional rounding according to significant figures of the numerical value. In addition, the phrase “about ‘x’ to ‘y’” includes “about ‘x’ to about ‘y’”.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range.

As used herein, the term “subject,” “patient,” or “organism” includes humans and mammals (e.g., mice, rats, pigs, cats, dogs, and horses). Typical hosts to which an agent(s) of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

The phrases “connected to” and “coupled to” refer to any form of interaction between two or more entities, including mechanical, electrical, magnetic, electromagnetic, fluid, and thermal interaction. Two components may be connected or coupled to each other even though they are not in direct contact with each other. For example, two components may be coupled to each other through an intermediate component.

The directional terms “proximal” and “distal” are used herein to refer to opposite locations on a device. The proximal end of the device is defined as the end of the device closest to the practitioner when the device is in use by the practitioner. The distal end is the end opposite the proximal end, along the longitudinal direction of the device, or the end furthest from the practitioner.

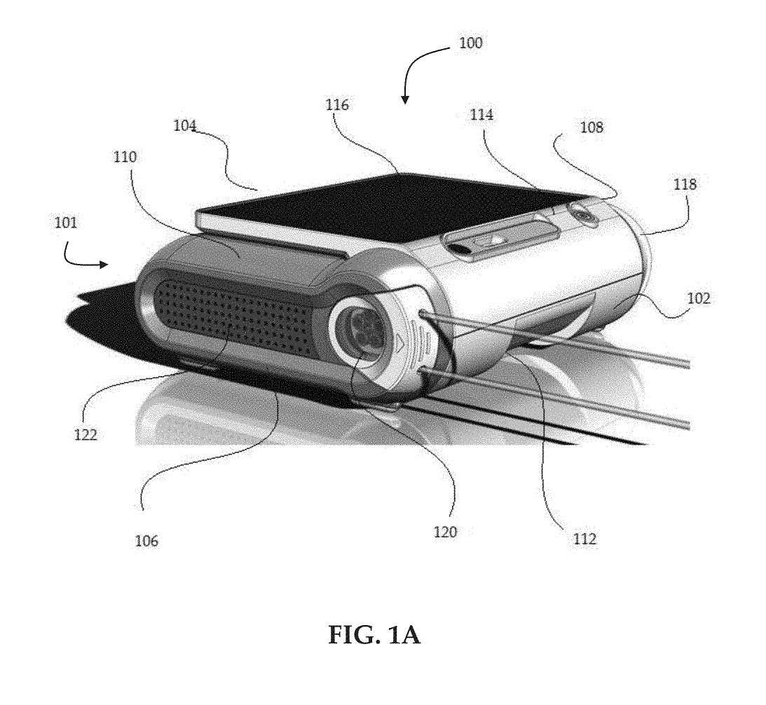

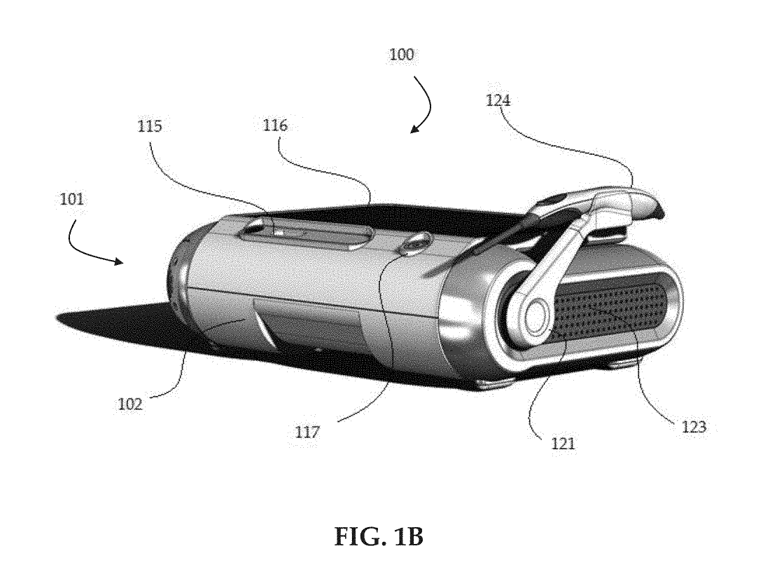

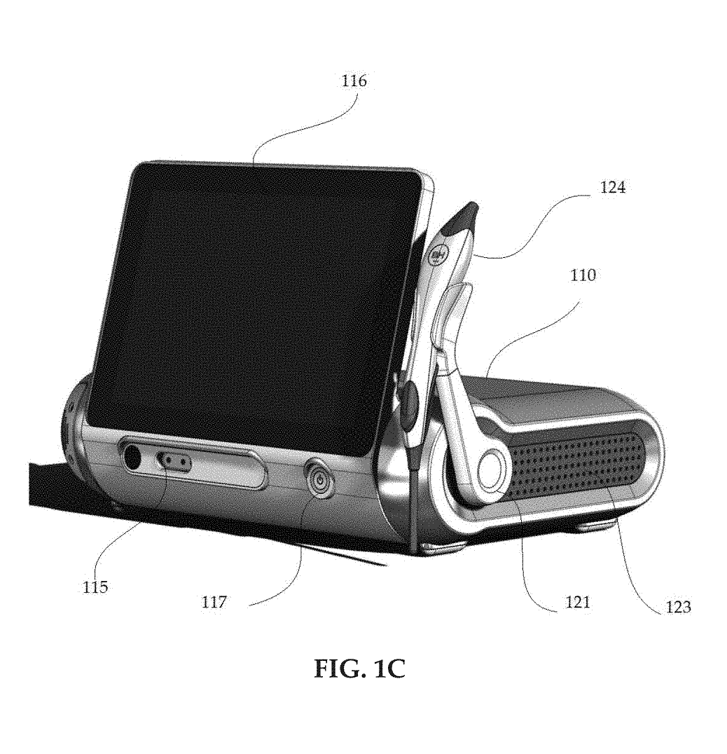

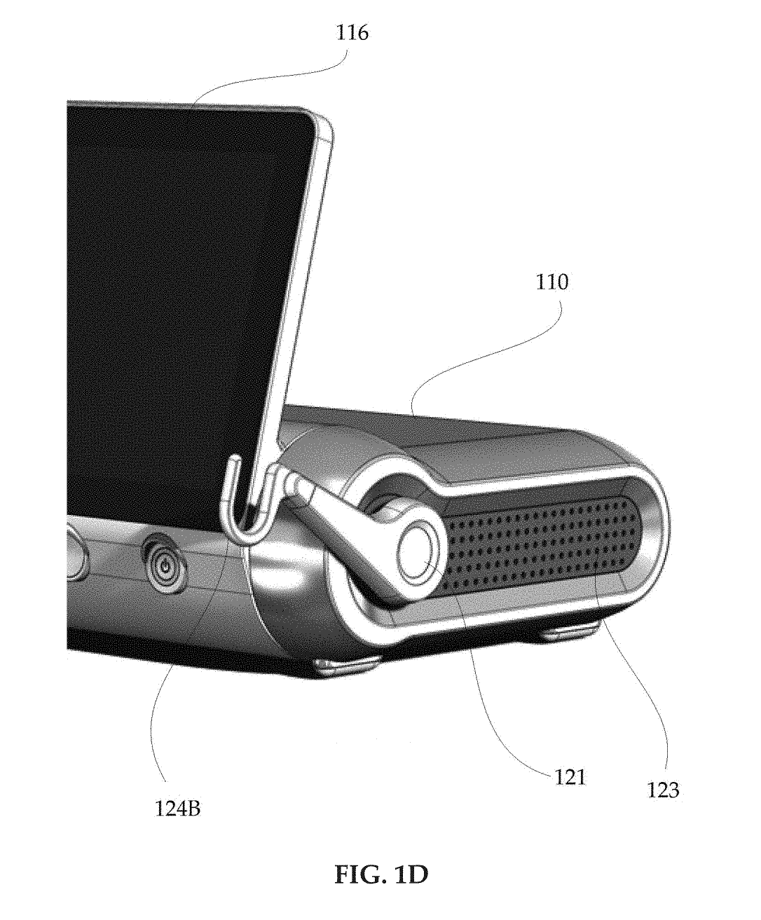

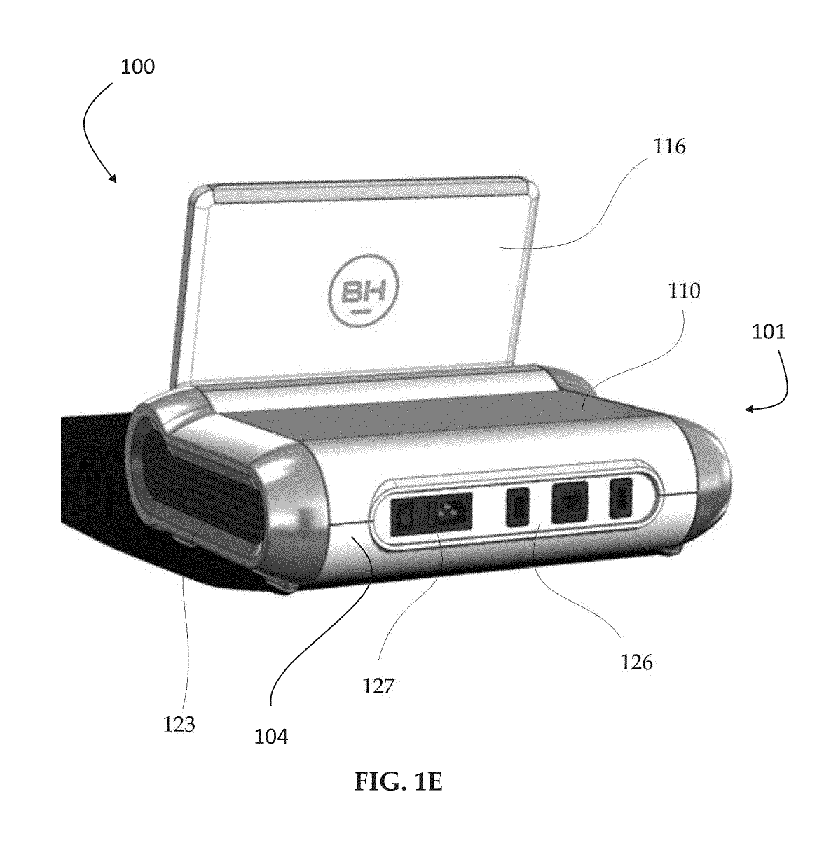

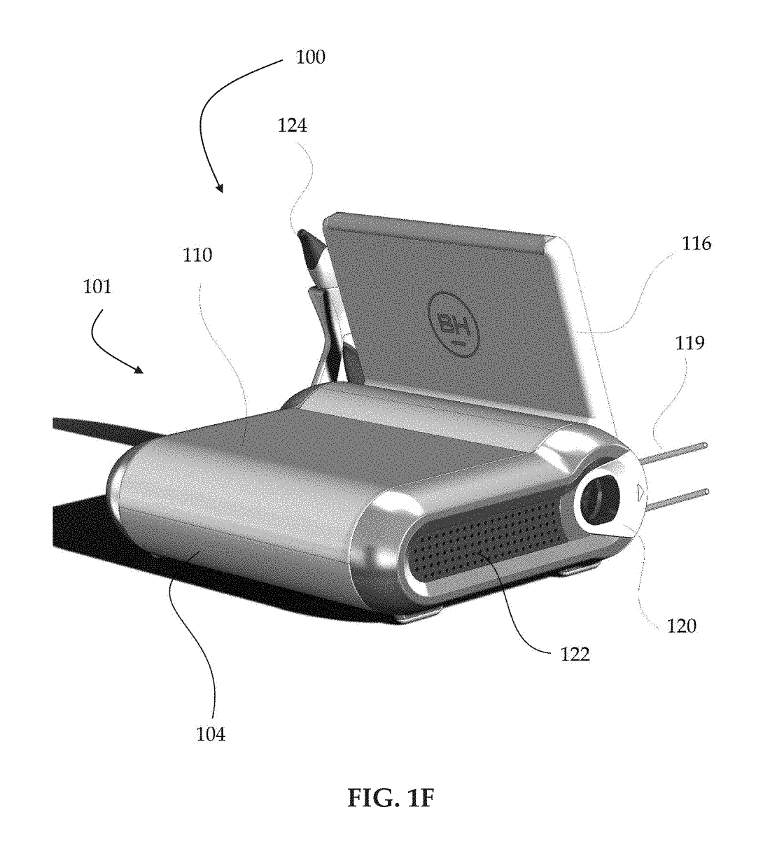

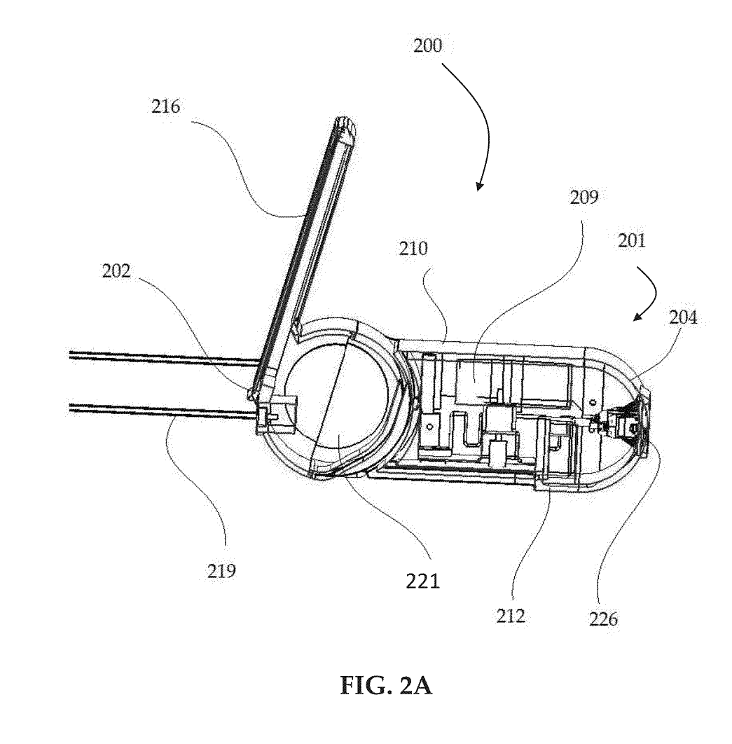

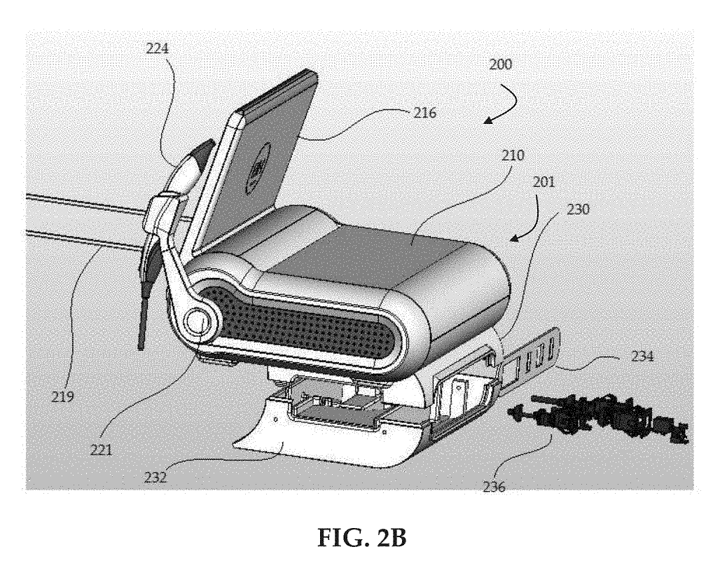

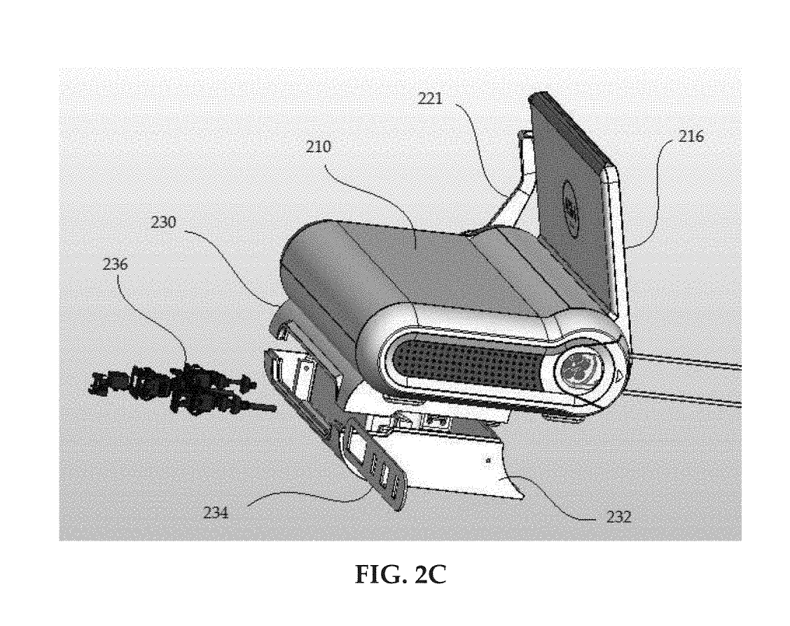

Referring in general to the following description and accompanying drawings, various embodiments of the present disclosure are illustrated to show its structure and method of operation. Common elements of the illustrated embodiments may be designated with similar reference numerals. Accordingly, the relevant descriptions of such features apply equally to the features and related components among all the drawings. Any suitable combination of the features, and variations of the same, described with components illustrated in FIG. 1, can be employed with the components of FIG. 2, and vice versa. This pattern of disclosure applies equally to further embodiments depicted in subsequent figures and described hereinafter. It should be understood that the figures presented are not meant to be illustrative of actual views of any particular portion of the actual structure or method but are merely idealized representations employed to more clearly and fully depict the present invention defined by the claims below.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

In an embodiment, the current invention is a modular medical console system/assembly that is adaptable for different medical functions/application, thus balancing cost versus performance of the console. As opposed to the conventional art, the console system is time- and cost-efficient, spreading non-recurring engineering costs over larger overall volumes, shared by a greater number of customers. The modular system provides applicability of a singular system across many customers and functions, as needed, optimizing cost efficiency and usability.

Console

As can be seen in FIGS. 1A-1F and 2A-2C, the current invention is a modular medical console 100 including an electro-mechanical housing. FIG. 1A depicts a housing 101 having a front side 102, rear side 104, left side 106, right side 108, top side 110, and bottom side 112. It should be understood that these terms are relative in nature and are used herein in a non-limiting fashion to discuss structural features of the pedestal stand. Throughout this disclosure the terms front side, front, and proximal are all interchangeable. Throughout this disclosure the terms back side, back, and distal are all interchangeable.

A top side 110 and/or front side 102 of the housing 101 includes a connector module 114 for a display device 116 (e.g., tablet), where the connector module 114 is coupled with a hinge 118 to the housing 101. When a display device 116 is coupled to the connector module 114, the display device can hinge/pivot between a substantially horizontal position (see FIGS. 1A-1B) that is parallel to the housing 101 and a substantially vertical position (see FIGS. 1C-1F) that is perpendicular to the housing 101, and any position therebetween or therebeyond. In the substantially horizontal position, the display device 116 may lie flat against or otherwise abut the top surface 110 of the housing 101 of the console 100. In a position between substantially horizontal and substantially vertical, an acute angle formed between the display device 116 and the top side 110 of the housing 101. In a position beyond substantial vertical, an obtuse angle is formed between the display device 116 and the top side 110 of the housing 101. The rotating position of the display device 116 permits use in different environments, for example on a tabletop, rack mounted, as vertical mounting for bed transport, etc. (not depicted).

In certain embodiments, depicts in FIGS. 1B-1D, the console includes one or two removable modular adjustable arms (such as 120 and 121) disposed on a left or right side of the housing 101. The arm holder/attachment (such as 120 or 121) is rotatable, as seen between FIGS. 1B and 1C, and may have any suitable structure for use for any suitable function, for example holding a handpiece (FIGS. 1B-1C), a medication bag (structure is a hook, as in FIG. 1D), and a patient tube-set, among others. This arm holder/attachment may be changed as needed.

In some embodiments a first modular device arm 120 can be attached to the housing 101 on the left side 106 of the console. When on the left side 106 the first modular device arm 120 is attached to the housing 101 close to left vents 122 built into the housing 101. In some embodiments of a modular medical console 100 there can be a second modular device arm 121 attached to the housing 101 the right side 108 of the housing 101. The second modular device arm 121 is attached to the housing 101 close to the right vents 123 built into the housing 101. The second modular device arm 121 is capable of holding a first medical tool 124. The first medical tool 124 may be a diagnostic medical device or a therapeutic medical device. In some embodiments the first medical tool 124 is any device used in a hospital or clinical setting that can be used in the care of patients.

The left and ride sides of the housing 101 include these integrated, recessed side-venting surfaces (i.e. left vents 122 and right vents 123), which provide airflow and thermal ventilation, without vertical fluid intrusion. However, these recessed sides including the left vents 122 and right vents 123 may be removable and replaced by solid sides (not depicted), depending on the ultimate use of the module.

In some embodiments the housing 101 near the display device 116 can have a first port 115. The first port 115 can be any combination of input or output ports to attach the modular medical console 100 to other medical devices, health or vitals monitoring systems, or other modular medical consoles. The port 115 are customizable and modular (i.e., removable and interchangeable) to accommodate varied uses of the module. The port 115 may be removed, as depicted in FIGS. 1A and 1B. In addition, in some embodiments the housing 101 has a power button 117 which can control the power to the display 116 or the first medical tool 124, or any combination of systems in the modular medical console 100. In alternative embodiments the first medical tool 124 is replaced with a second medical tool 124B depicted in FIG. 1D, which can be a hook to either help organize wires or attach the modular medical console 100 to a wall, pole, or to another console.

In some embodiments the modular medical console 100 there is no first modular device arm 120 or second modular device arm 121 as depicted in FIG. 1E. The right vents 123 would extend from the rear side 104 to the front side 102 of the housing 101 as depicted in FIG. 1E. The back side of display device 116 is also depicted in FIG. 1E. In some embodiments, the rear side 104 of housing 101 has a second port 126. The second port can be any combination of input or output ports to attach the modular medical console 100 to other medical devices, health or vitals monitoring systems, or other modular medical consoles. The second port 126 is customizable and modular (i.e., removable and interchangeable) to accommodate varied uses of the module. The second port 126 may be removed as depicted in FIG. 1F. In some embodiments the second port 126 has a power port 127. Power port 127 has a port to which a power cord can be attached to the modular medical console 100 to provide power to the console. In addition, the power port 127 may have a power switch to control power in the modular medical console 100.

Furthermore, the modular medical console 200 may include generic connector mounting panels depicted in FIGS. 2B and 2C such as top mounting panel 230 and bottom mounting panel 232. These mounting panels permit customizable connections depending on end-user needs by modifying the connector panels to accommodate the required connectors (e.g., dedicated handpiece cables, discrete switches, USB data-ports, remote display, power entry, etc.). The top mounting panel 230 and bottom mounting panel 232 can come together with a rear mounting panel 234 which can include any number of connectors 236.

In some embodiments the rear side 104 of housing 101 does not have any additional ports, as depicted in FIG. 1F. The back side of display 116 and the top side of the housing 101 is depicted. In addition, in some embodiments a first modular device arm 120 can be attached to the housing 101 on the left side 106 of the console. When on the left side 106 the first modular device arm 120 is attached to the housing 101 close to left vents 122 built into the housing 101. In some embodiments the first modular device arm 120 also has cables 119 which can link the modular medical console 100 to other devices, monitoring equipment, or other modular medical consoles. The second modular device arm 121 is attached to the housing 101 close to the right vents 123 built into the housing 101. The second modular device arm 121 is capable of holding a first medical tool 124. The first medical tool 124 may be a diagnostic medical device or a therapeutic medical device. In some embodiments the first medical tool 124 is any device used in a hospital or clinical setting that can be used in the care of patients.

In another embodiment, the modular medical console 200 has a housing 201 with a front side 202, top side 210, bottom side 212, and rear side 204. The rear side 204 has a second port 226. In some embodiments the second modular device arm 221 is attached to the housing 201 close to the right vents (not depicted) built into the housing 201. FIG. 2A shows a schematic of modular medial console 200 and depicts the inside 209 of the housing 201. The display device 216 is coupled with a hinge to housing 201. In some embodiments the first modular device arm (not depicted in FIG. 2A) also has cables 219 which can link the modular medical console 200 to other devices, monitoring equipment, or other modular medical consoles.

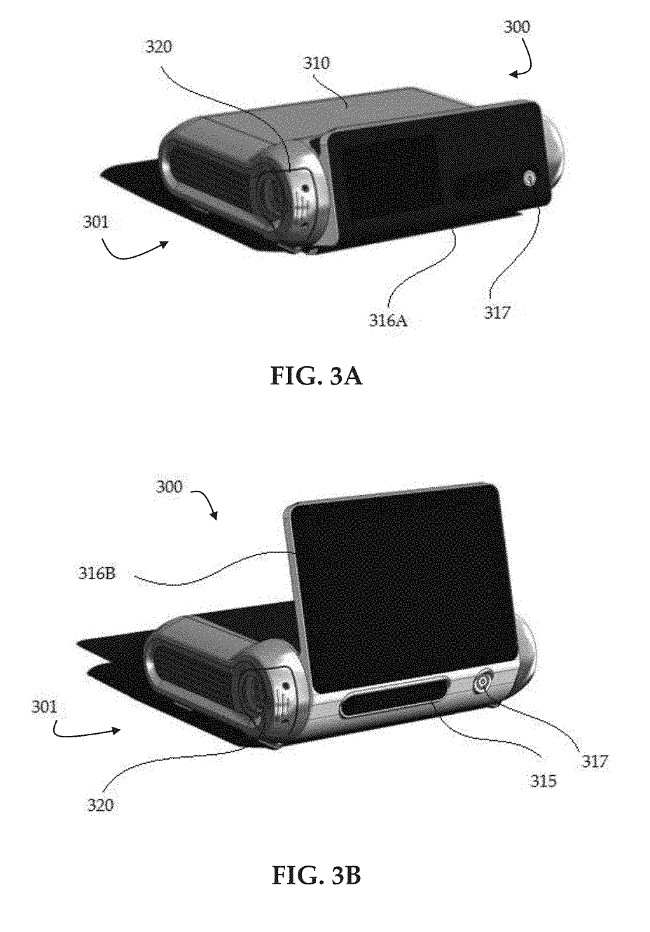

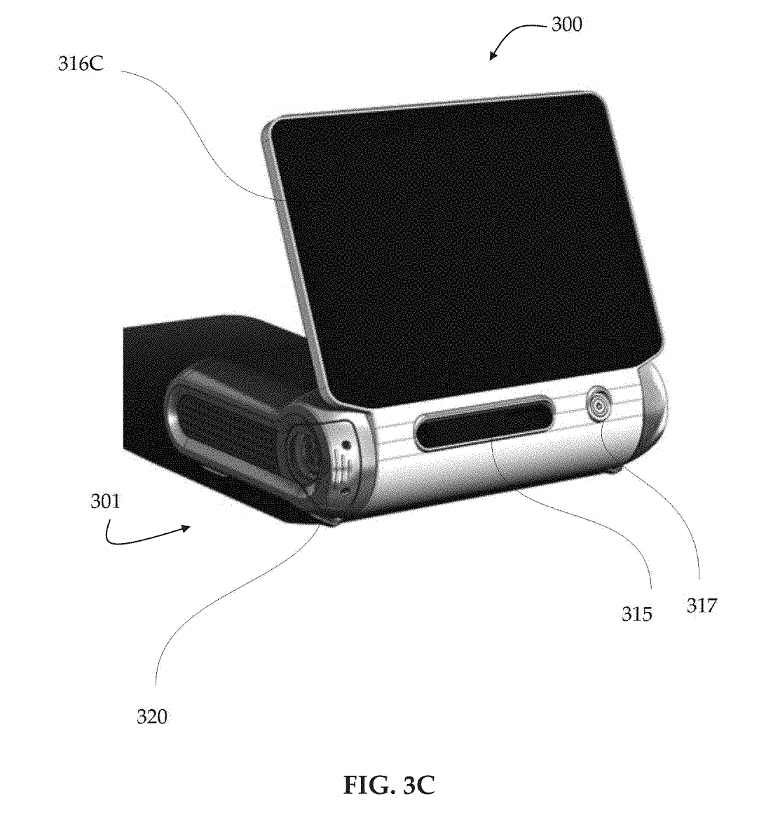

In certain embodiments, the display device coupled to the console can be any suitable device, for example a 6″ screen and status bar is the display device 316A depicted in FIG. 3A, a 12″ screen is the display device 316B depicted in FIG. 3B, and a 16″ screen is the display device 316C depicted in FIG. 3C. Any portion of the front of the modular medical console 300 can be removable to accommodate differing devices. For instance, the embodiment of FIG. 3A includes an entire front block component on housing 301 that includes the display device 316A. That block component is rotatable and removable to accommodate other block components that can be coupled to the screens of FIGS. 3B-3C, for example. In these figures, the display screen is affixed to the block component, though it is contemplated that the block component can include a connector that can electrically couple multiple displays of different sizes. In some embodiments the modular medical console 300, will have a built-in display device 316A that may be an LCD display or other suitable technology that receives display data from a computer within the console. In some embodiments, the display device 316A is an integrated computer, such as a tablet, that receives incoming data from components elsewhere on the modular medical console 300.

In certain embodiments, the current console can be used for a variety of functions, for example including, but not limited to, operating room, intensive care unit, and bedside environments for therapies such as ventilation, dialysis, and IV; surgical procedures involving energy-bases such as RF, plasma, and ultrasound, suction and aspiration systems.

Example Application

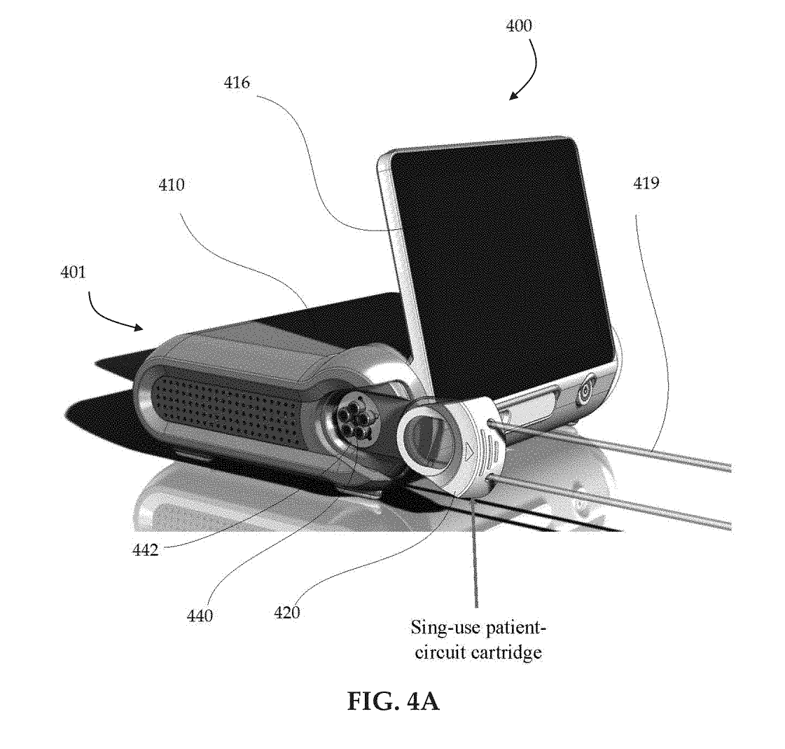

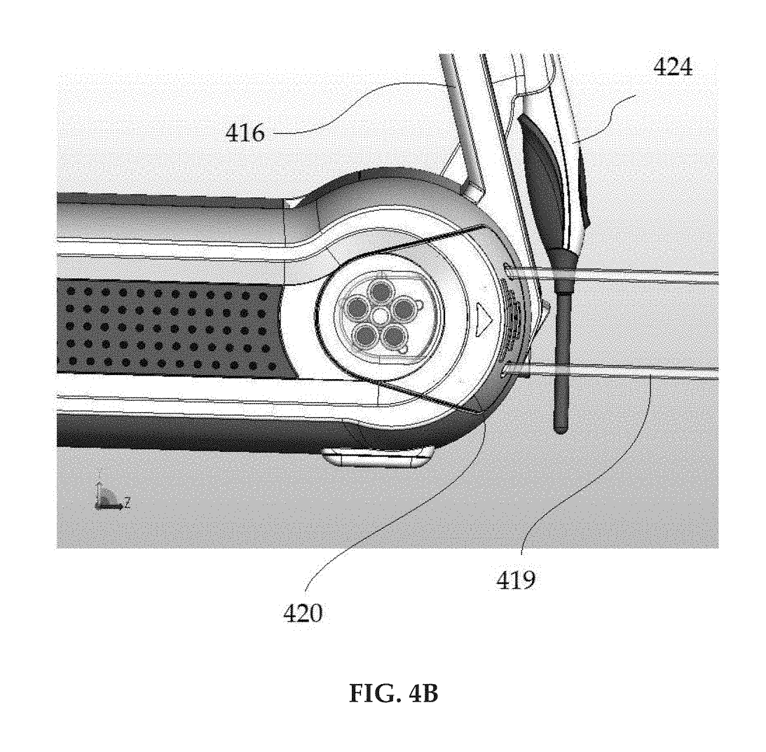

FIGS. 4A and 4B depict an exemplary application or implementation of the modular medical console 400, with display device 416, first medical tool 424, also includes a peristaltic pump with single-use patient-circuit cartridge 420, used for any medical purpose, such as IV fluid delivery or blood circulation. The patient-circuit cartridge 420 includes the patient-circuit cartridge which holds the tubing and the flexible tubing 419, through which fluid flows. When the patient-circuit cartridge 420, with flexible tubing 419 secured thereto, is installed onto the housing 401 as depicted in FIG. 4A and FIG. 4B, the flexible tubing 419 is disposed around a set of rollers 440 extending from or disposed along the circumference of the rotor 442 of a peristaltic pump. In some embodiments two patient-circuit cartridges (not depicted) can be integrated to the modular medical console 400 on both sides of the housing 401. In some embodiments the patient-circuit cartridge 420 may be integrated to either the left or right side of housing 401.

After use of the patient-circuit cartridge, it may be disposed.

Pedestal Stand

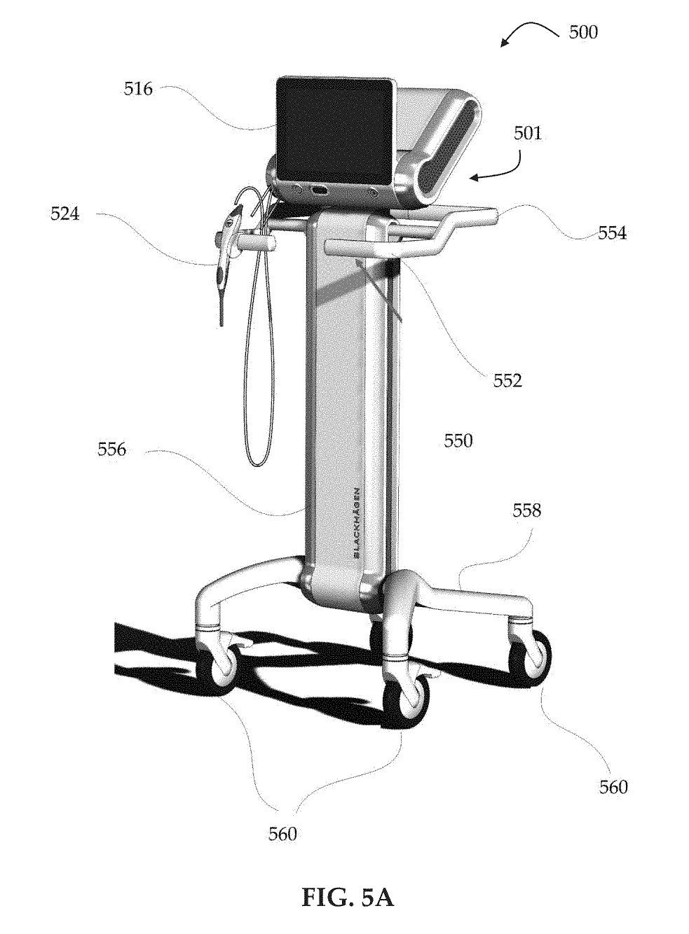

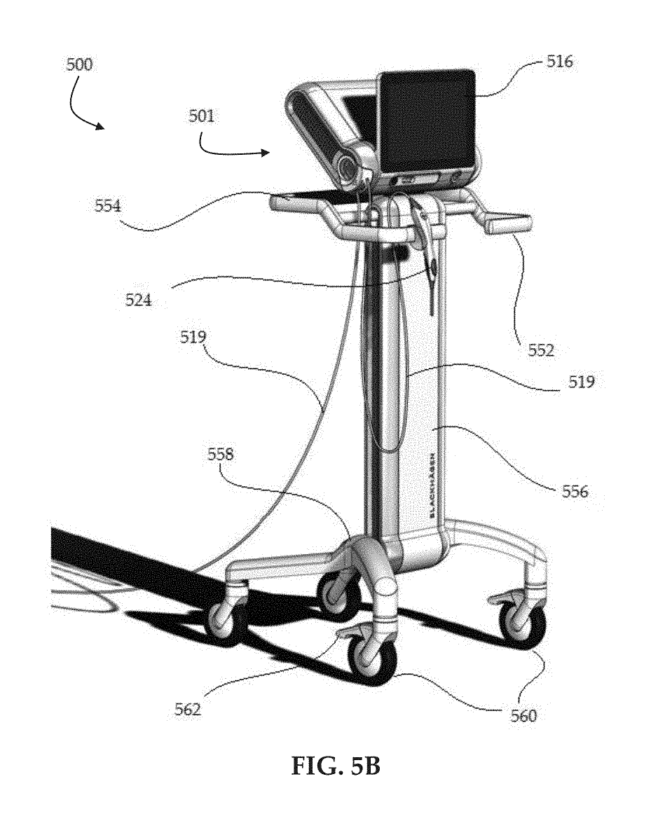

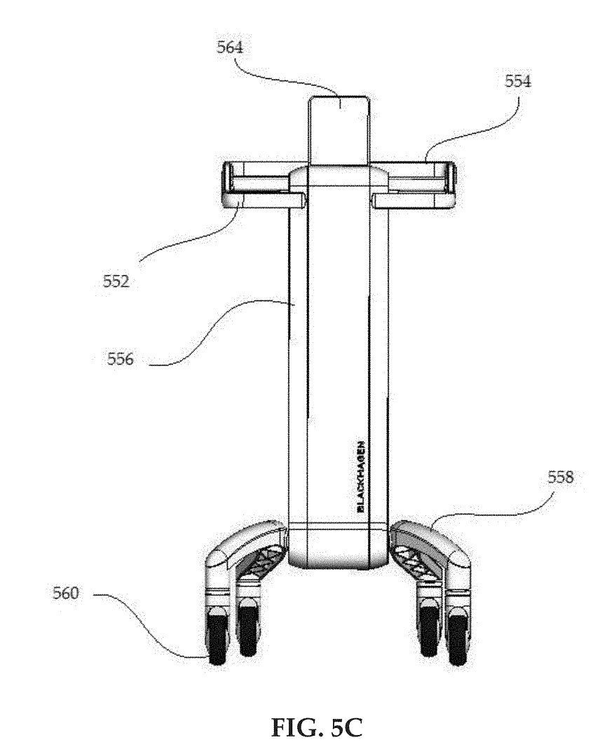

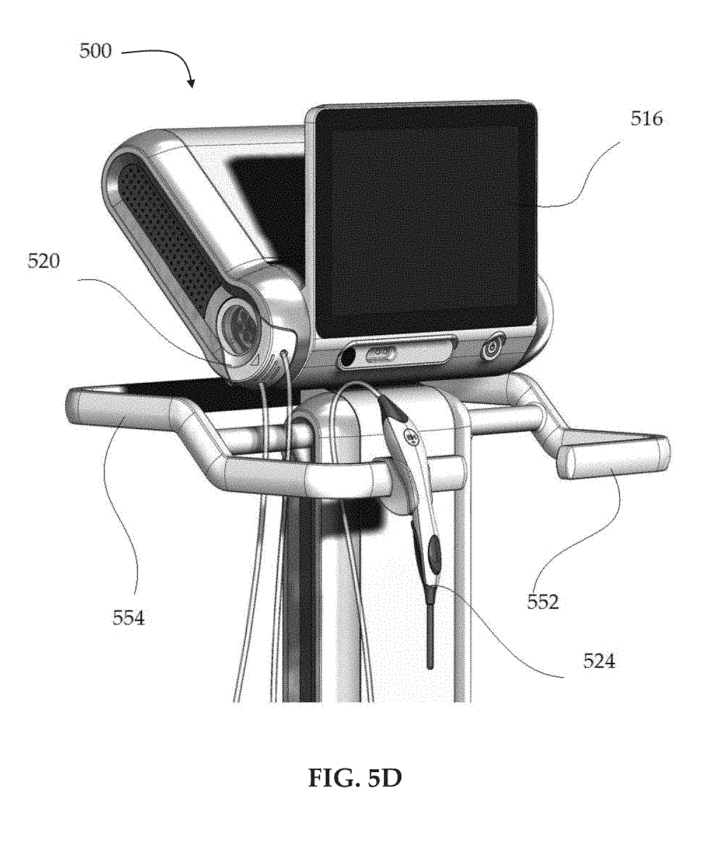

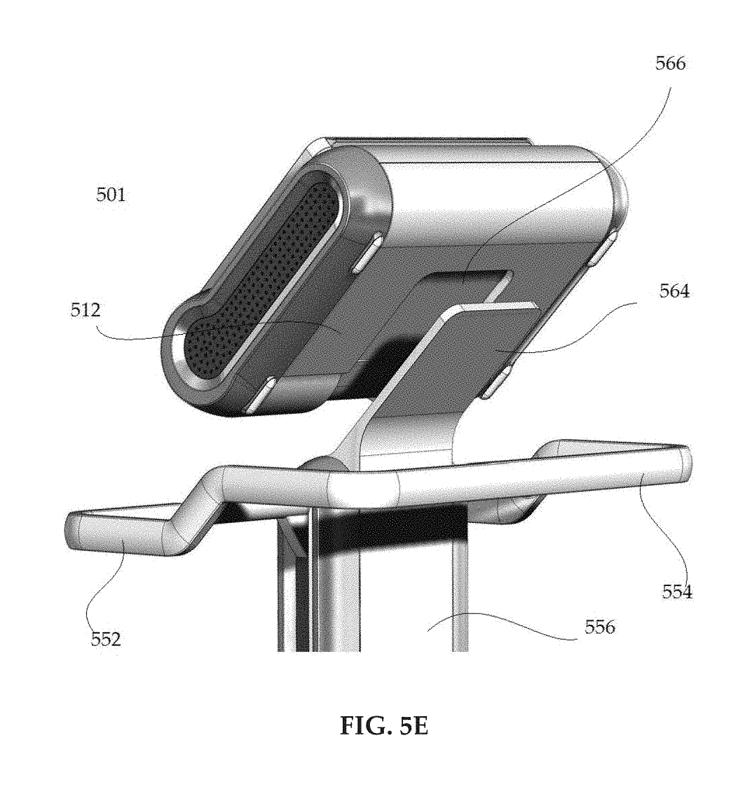

In certain embodiments, as seen in FIGS. 5A-5E, the modular medical console 500 further includes a dedicated rolling pedestal stand 550 that secures to the bottom side of the housing 501. The modular medical console 500 includes a display device 516 and first medical tool 524. The rolling pedestal stand 550 includes proximal handles 552, distal handles 554, upright 556 and base 558. The base 558 has wheels 560 coupled to it in some embodiments. As depicted in FIG. 5B the first medical tool 524 with attaching cables 519 is coupled to the housing 501. In addition, the wheels 560 may also be controlled with a braking system 562. The modular medical console 500 is easily removable from the pedestal stand 550 via a tool-less interface or connector 564 that connects to the bottom side 512 of housing 501 into recess 566 as depicted in FIG. 5C and FIG. 5E.

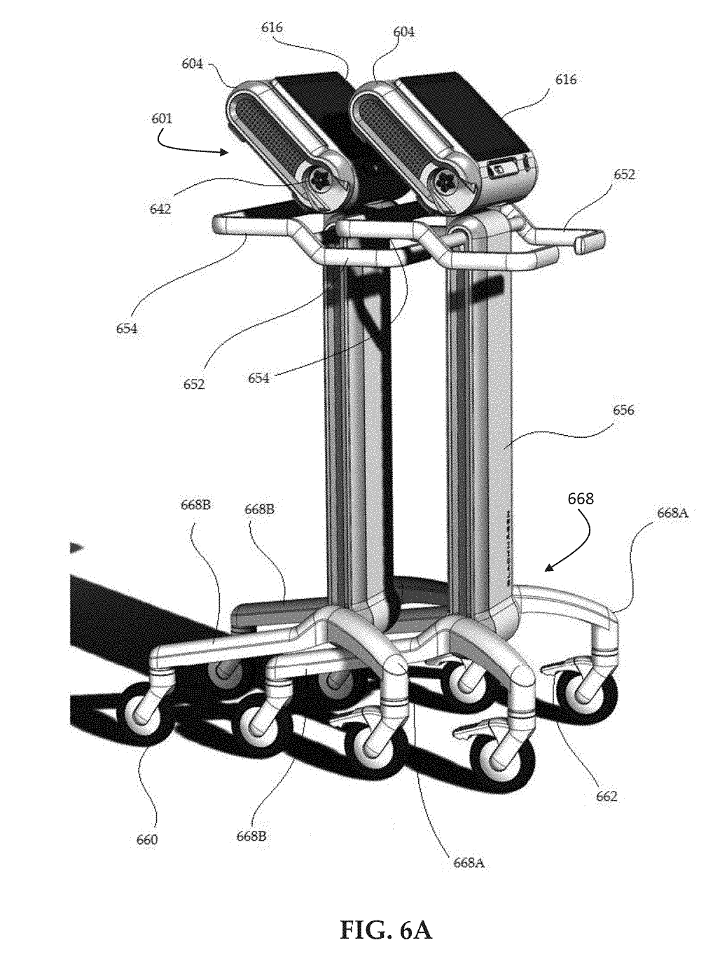

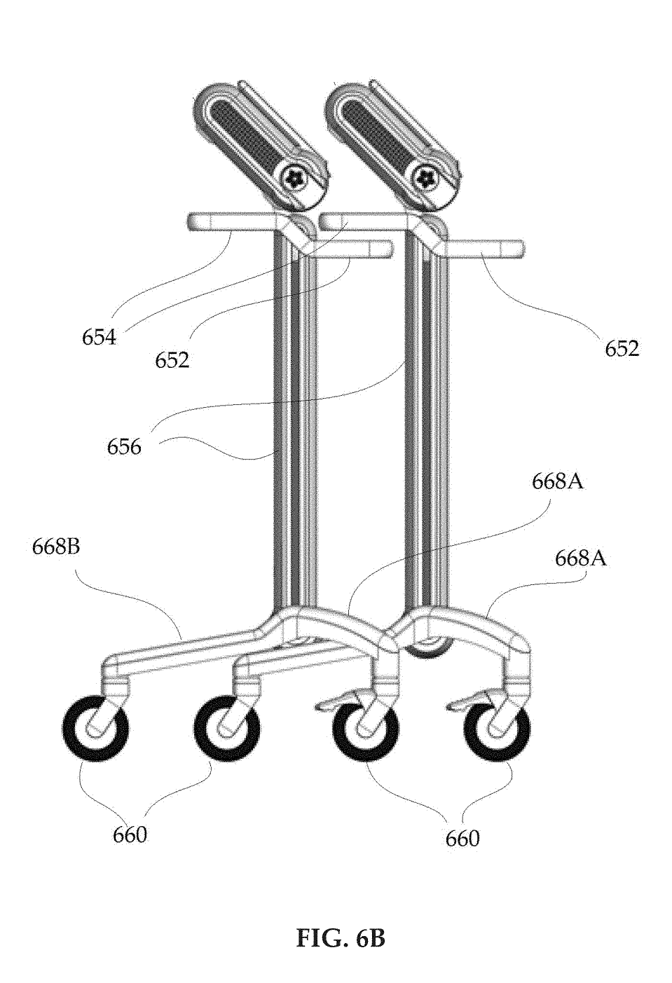

Further, FIGS. 6A-6B show that the pedestal stand is structured in a specific manner such that multiple pedestal stands may be nested together to maximize storage efficiency, in turn enabling multi-unit transportation/deployment and grouped charging of any onboard batteries. FIG. 6A and FIG. 6B depicts two pedestal stands nested together. The housing 601 with rear side 604 and display device 616 fit next to each other when the pedestals are nested. Each pedestal has proximal handles 652 which fits under distal handles 654 when the pedestals are nested. The pedestal stand 656 is also coupled to pedestal base 668 which has proximal pedestal base 668A and distal pedestal base 668B. The pedestal base is coupled to wheels 660 which can be controlled with a braking system 662. The proximal pedestal base 668A fits over the distal pedestal base 668B as depicted in FIG. 6A. Nested pedestal stands also provides a significantly denser storage arrangement of many of the same devices, reducing storage footprint requirements. Nesting, therefore, also facilitates group charging via daisy-chain connection to a charging source.

The pedestal stand includes a central, thin upright 656 having a top side, bottom side, left side, right side, front side and rear side. It should be understood that these terms are relative in nature and are used herein in a non-limiting fashion to discuss structural features of the pedestal stand.

The pedestal stand further includes a pair of proximal pedestal bases 668A and wheels 660 extending in front of the upright 656 and a pair of distal pedestal bases 668B and wheels 660 extending to the rear of the vertical column. As can be seen most clearly in FIG. 5C, the front set of wheels 560 extend further laterally than the rear set of wheels. In other words, a greater distance is present between the front wheels than between the rear wheels. The proximal pedestal bases 668A have a generally greater height or curvature than the distal pedestal bases 668B.

This structural configuration results in the ability for multiple pedestal stands to nest with one another with respect to their legs, as can be seen in FIGS. 6A-6B. Specifically, as depicted, when nesting two pedestal stands together, the front wheels 660 of a rearward pedestal stand roll past the rear wheels 660 of a frontward pedestal stand, to the outside of the rear wheels of the frontward pedestal stand. Subsequently, the proximal pedestal base 668A of the rearward pedestal stand slide in overlying to the rear wheels 660 of the frontward pedestal stand. In this manner, the pedestal bases 668 of the two pedestal stands are able to nest with one another.

It can be appreciated that an opposite structural configuration—rear wheels extend further laterally than front wheels, and rear legs are higher than front legs—is contemplated herein, as it would provide a similar functionality.

Now referring back to FIGS. 5A-5E, the pedestal stand further includes one or more handles disposed substantially around the upright 556 or at least partially in front of the column and behind the column. For example, the figures depict a single handle connected to and disposed around the upright 556. An empty space can be seen in the portion in the handle in front of the column, but the handle is still considered herein to be “substantially around” the column. It can be appreciated that this handle can be split into two handles, a proximal handle 552 and distal handle 554, as long as one handle is disposed at least partially in front of the upright 556 and one handle is disposed at least partially in the rear of the upright 556. The handle will be referred to herein as the “front handle” or “proximal handle” and the “rear handle” or “distal handle,” though it is appreciated that this may form multiple handles, or a single handle disposed substantially around the upright. As can be seen the clearest in FIGS. 5C-5E, the rear handle is positioned at a greater height than the front handle. In alternative embodiments, the front handle can be positioned at a greater height than the rear handle.

This structural configuration results in the ability for multiple pedestal stands to nest with one another with respect to their handles, as can be seen in FIGS. 6A-6B. Specifically, as depicted, when nesting two pedestal stands together, the front handle of the rearward pedestal stand is disposed in underlying relation to the rear handle of the frontward pedestal stand.

It can be appreciated that an opposite structural configuration—front handle positioned higher than rear handle—is contemplated herein, as it would provide a similar functionality. Generally, differing front and rear handle heights facilitate nesting.

Furthermore, the front-to-back thinness of the upright 556 permits maximum nesting efficiency when multiple pedestal stands are positioned adjacent to each other.

Optionally, the pedestal stand may include integrated mounting components (not depicted) on the underside of the vertical column and/or along the left and right sides of the vertical column, thus permitting accessory brackets for connection to various accessories in front of and/or behind the pedestal stand. The mounting interface also allows for daisy-chain charging connection of multiple nested devices.

Different Implementations

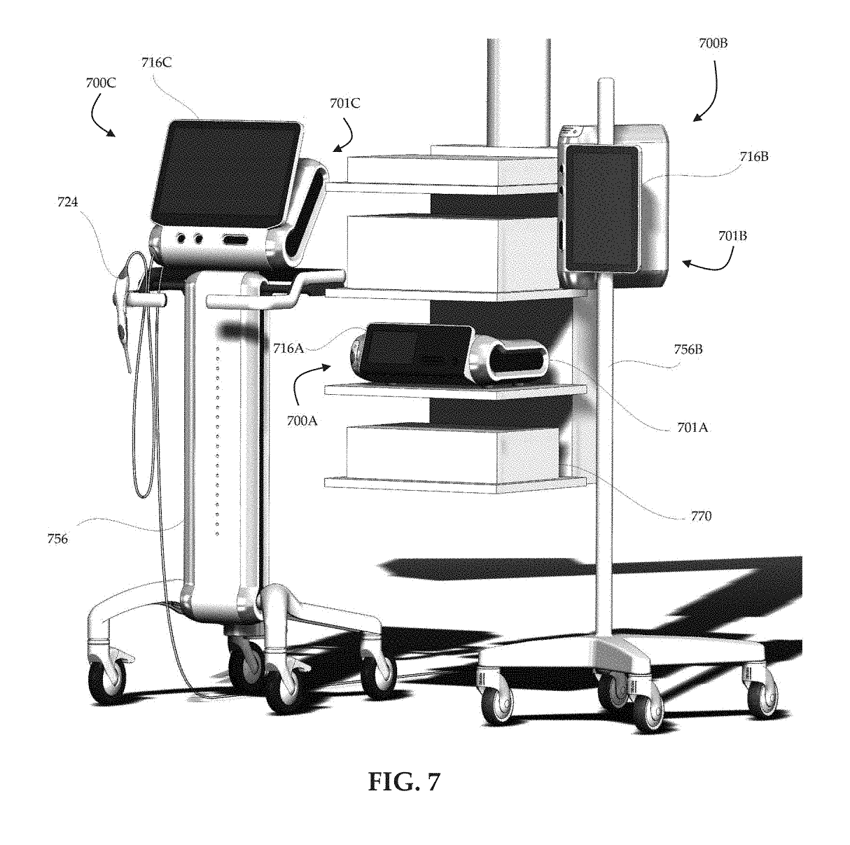

FIG. 7 depicts different display devices 716A, 716B, and 716C that may be used with the current console, as discussed herein and also seen in FIGS. 3A-3C. FIG. 7 further depicts different implementations of said console. As examples, the console can be used with housing 701C with display device 716C couple to the pedestal with upright 756, on a tabletop with other miscellaneous items 770, where the modular medical console 700A has housing 701A with display device 716A, and on an IV stand/pole 756B. The console may be coupled to a variety of medical devices for ease of use and multifunctionality.

In some embodiments nesting and interlocking the pedestals enables multiple devices to be conveniently transported by a single end-user. In this embodiment the devices can be moved simultaneously and deployed as needed.

The advantages set forth above, and those made apparent from the foregoing description, are efficiently attained. While the disclosure is susceptible to various modifications and implementation in alternative forms, specific embodiments have been shown by way of non-limiting example in the drawings and have been described in detail herein. Since certain changes may be made in the above construction without departing from the scope of the instant application, it is intended that all matters contained in the foregoing description or shown in the accompanying drawings shall be interpreted as illustrative and not in a limiting sense.

The disclosure is not intended to be limited to the particular forms disclosed. Rather, the disclosure is to cover all modifications, equivalents, and alternatives falling within the scope of the disclosure as defined by the following appended claims and their legal equivalents.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art, and having the benefit of this disclosure, that changes may be made to the details of the above-described embodiments without departing from the underlying principles of the disclosure herein.

It is also to be understood that the following claims are intended to cover all of the generic and specific features of the invention herein described, and all statements of the scope of the invention that, as a matter of language, might be said to fall therebetween.

What is claimed is:

1. A modular medical console, comprising:
a housing having a front end, a rear end, a left side, a right side, a top side, and a bottom side;
a display device connector module that is hingedly connected to the front end of the housing, wherein,
a display device is connected to the display device connector module and when the display device has a substantially horizontal position it abuts or is otherwise parallel to the top side of the housing,
the display device connected to the display device connector module has an angled position relative to the top side of the housing, such that the angle between the display device and the top side of the housing is more than 0° and less than 360°; and
a plurality of modular components removably coupled to the housing.

2. The modular medical console of claim 1, wherein the modular medical console is portable.

3. The modular medical console of claim 1, wherein the housing is coupled to a pedestal.

4. The modular medical console of claim 3, wherein the pedestal further comprises handles coupled to an upright, wherein the upright is coupled to pedestal base which itself is operably coupled to a set of wheels.

5. The modular medical console of claim 3, wherein the housing is coupled to the pedestal with a toolless connection system.

6. The modular medical console of claim 1, wherein the housing further comprises one or two removable modular adjustable arms disposed on either the left or right side of the housing, wherein one or both of the two removable modular adjustable arms has any one of a sensor interface, filter element, UV disinfection treatment device, diagnostic tool, or therapeutic tool attached to the removable modular adjustable arms.

7. The modular medical console of claim 6, wherein one or both of the removable modular side adjustable arms is coupled to an integrated peristaltic pump, wherein the peristaltic pump includes a rotor, rollers, and a patient-circuit cartridge that is removably disposed in overlying relation to the rotor and rollers; and wherein one or both of the removable modular adjustable arms may also integrate other functional features such as sensors, filters, and disinfection generators.

8. The modular medical console of claim 6, wherein at least one of the removable modular adjustable arms is a hook holding an object selected from the group consisting of a handpiece, a medication bag, and a patient tube-set.

9. The modular medical console of claim 6, wherein at least one of the removable modular adjustable arms holds a first medical tool.

10. The modular medical console of claim 9, wherein the first medical tool is selected from the group consisting of a diagnostic medical device and a therapeutic medical device.

11. The modular medical console of claim 1, wherein the housing further comprises a port.

12. The modular medical console of claim 11 wherein the port is an input/output panel disposed on the front or rear side of the housing.

13. The modular medical console of claim 11, wherein the housing comprises a second port.

14. The modular medical console of claim 1, further comprising vents on the side of the housing.

15. The modular medical console of claim 1, further comprising a generic connector mounting panel coupled to the bottom side of the housing to mount the housing on the generic connector mounting panel.

16. The modular medical console of claim 15, wherein the generic connector mounting panel includes a top mounting panel and a bottom mounting panel, wherein the top mounting panel and the bottom mounting panel are coupled together with a rear mounting panel and one or more connectors.

17. The modular medical console of claim 3, wherein the pedestal comprises a thin, vertical upright column having a front side, a rear side, a left side, a right side, a top end, and a bottom end; wherein the bottom end is coupled to a base; and wherein the vertical upright column has a battery pack or other component held within the vertical upright column.

18. The modular medical console of claim 17, wherein the base includes a pair of front legs and wheels extending from the vertical upright column toward the front of the pedestal stand;
a pair of rear legs and wheels extending from the vertical upright column toward the rear of the pedestal stand,
wherein the pair of front wheels extend further laterally than the pair of rear wheels, such that a greater distance is present between the pair of front wheels than between the pair of rear wheels,
wherein the pair of front legs have a greater height or curvature than the pair of rear legs,
wherein the pair of rear legs and wheels is configured to nest with a second pair of front legs and wheels of a second pedestal stand, whereby the second pair of front legs and wheels of the second pedestal stand is structured similarly to the pair of front legs and wheels of the pedestal stand.

19. The modular medical console of claim 17, wherein the pedestal further comprises a proximal handle coupled to the vertical upright column and positioned to the front of the vertical upright column, wherein the proximal handle has a first height off the ground on which the pedestal stand is disposed, a distal handle coupled to the vertical upright column and positioned to the rear of the vertical upright column, wherein the distal handle has a second height off the ground on which the pedestal stand is disposed, wherein the second height is greater than the first height, wherein the proximal handle and the distal handle are formed of multiple handles or are formed of a single handle disposed substantially around the vertical upright column; and wherein the pedestals nest together to take up less storage space.

20. A method of using a modular medical console to be used in patient care, comprising;
proving a modular medical console, comprising:
a housing having a front end, a rear end, a left side, a right side, a top side, and a bottom side;
a display device connector module that is hingedly connected to the front end of the housing, wherein,
a display device is connected to the display device connector module and when the display device has a substantially horizontal position it abuts or is otherwise parallel to the top side of the housing,
the display device connected to the display device connector module has an angled position relative to the top side of the housing, such that the angle between the display device and the top side of the housing is more than 0° and less than 360°; and
a plurality of modular components removably coupled to the housing
wherein the modular medical console is used in patient care.
